(19) United States
(12) Patent Application Publication
(10) Pub. No.: US 2025/0295580 A1
(43) Pub. Date: Sep. 25, 2025

(54) LAYERED POLYMERIC COATINGS FOR DRUG RELEASE

(71) Applicant: Becton, Dickinson and Company, Franklin Lakes, NJ (US)

(72) Inventors: Brendan Laine, Draper, UT (US); Nicholas Papadopoulos, South Salt Lake, UT (US); Zachary Hales, Stansbury Park, UT (US); James Freasier, Salt Lake City, UT (US)

(21) Appl. No.: 18/610,053

(22) Filed: Mar. 19, 2024

(51) Int. Cl. A61K 9/00; A61M 25/00

(52) U.S. Cl. CPC A61K 9/0024; A61M 25/0045

(57) ABSTRACT

A medical device may include a medical device surface, a primary coating on the medical device surface, and a top coating on the primary coating. The primary coating may include a coating matrix, and a water-soluble active pharmaceutical ingredient (API) within the coating matrix. The coating matrix may include a hydrophilic polymer, and the water-soluble API may be uniformly dispersed within the coating matrix. The top coating may include an inert polymer. In response to water crossing the top coating into the coating matrix, the water-soluble API may be released from the coating matrix and the primary coating may swell to create a pressure that drives the API across the top coating. A method of preparing layered polymeric coatings, including the top coating and the primary coating, on the medical device may include solvent-casting the medical device surface to form the primary coating and the top coating.

### BACKGROUND

Traditional drug-eluting polymeric coatings for medical devices primarily rely on diffusion-driven release mechanisms, utilizing Fickian dynamics from biodegradable or non-biodegradable polymers. These systems are designed to release a drug or active pharmaceutical ingredient (API) but often suffer from limitations such as weak elution, dependency on environmental stimuli (e.g., pH) for initiating release, or rapid API release leading to potential acute toxicity. Various approaches to control drug release have been explored, including the incorporation of drugs into polymer matrices or the application of a single coating on a medical device surface to modulate diffusion. However, these methods have faced challenges in achieving controlled, sustained release without initial burst release and in maintaining functionality across a wide range of API and polymer combinations.

An initial burst release can be problematic because a rapid release of API can lead to high concentrations of API in the human body, potentially causing toxic effects. Also, after the burst release, the remaining amount of API might not be sufficient to maintain therapeutic levels for an intended duration, leading to reduced efficacy of a treatment. Moreover, fluctuating API levels can result in a need for more frequent dosing or lead to periods of subtherapeutic API concentrations, both of which can negatively affect patient compliance and treatment outcomes.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to layered polymeric coatings for a medical device, as well as related methods. In some embodiments, a medical device, which may be implantable or indwelling, may include a medical device surface. In some embodiments, the medical device may include a primary coating on the medical device surface. In some embodiments, the primary coating may include a coating matrix and a water-soluble API within the coating matrix. In some embodiments, the coating matrix may include a hydrophilic polymer and the water-soluble API may be uniformly dispersed within the coating matrix to form a monolith.

In some embodiments, the medical device may include a top coating on the primary coating. In some embodiments, the top coating may include an outer most coating of the medical device surface. In some embodiments, the top coating may include an inert polymer. In some embodiments, the top coating may be hydrophobic or more hydrophobic than the primary coating. In some embodiments, the medical device may deliver sustained release of the water-soluble API by non-Fickian diffusion across the top coating. In further detail, in some embodiments, in response to water crossing the top coating into the coating matrix, the water-soluble API may be released from the coating matrix and the primary coating may swell to create a pressure that drives the water-soluble API across the top coating.

In some embodiments, the water-soluble API may be uniformly dispersed in the coating matrix such that the coating matrix and water-soluble API are monolithic. In some embodiments, the water-soluble API may not be covalently or ionically bound to the coating matrix.

In some embodiments, the inert polymer of the top coating may include polyethylene-co-vinyl acetate or another suitable hydrophobic resin.

In some embodiments, the coating matrix may include a non-ionic polyurethane. In some embodiments, the coating matrix may include an aromatic-polyether polyurethane, an aromatic-polycarbonate polyurethane, an aliphatic-polyether polyurethane, an aliphatic-polycarbonate polyurethane, or another suitable polyurethane.

In some embodiments, the primary coating and the top coating may be solvent-cast. In some embodiments, the primary coating may have a thickness of about 15 micrometers or at least 15 micrometers. In some embodiments, the top coating may have a thickness of less than 15 micrometers.

In some embodiments, a method of preparing layered polymeric coatings on the medical device to deliver sustained release of the water-soluble API by non-Fickian diffusion across the top coating may include obtaining the medical device, which may include the medical device surface.

In some embodiments, the method may include forming the primary coating on the medical device surface. In some embodiments, forming the primary coating on the medical device surface may include forming a first solution by dissolving a hydrophilic polymer and the water-soluble API in a first solvent such that the water-soluble API is uniformly dispersed within the coating matrix.

In some embodiments, forming the primary coating on the medical device surface may include solvent-casting the medical device surface in the first solution. In some embodiments, forming the primary coating on the medical device surface may include evaporating the first solvent from the medical device surface.

In some embodiments, the method may include forming the top coating on the primary coating. In some embodiments, forming the top coating on the primary coating may include dissolving the inert polymer in a second solvent to form a second solution. In some embodiments, forming the top coating on the primary coating may include solvent-casting the medical device surface having the primary coating on the medical device surface in the second solution. In some embodiments, forming the top coating on the primary coating may include evaporating the second solvent from the medical device surface.

In some embodiments, in response to water crossing the top coating into the coating matrix, the water-soluble API may be released from the coating matrix and the primary coating may swell to create the pressure to drive the API across the top coating.

In some embodiments, the first solvent may include a polar organic solvent. In some embodiments, the hydrophilic polymer may be non-ionic. In some embodiments, the second solvent may include a non-polar organic solvent. In some embodiments, the non-polar organic solvent may include toluene or another suitable non-polar organic solvent. In some embodiments, the inert polymer of the top coating may include polyethylene-co-vinyl acetate.

In some embodiments, the coating matrix may include a non-ionic polyurethane. In some embodiments, the coating matrix may include an aromatic-polyether polyurethane, an aromatic-polycarbonate polyurethane, an aliphatic-polyether polyurethane, or an aliphatic-polycarbonate polyurethane.

In some embodiments, the medical device surface may include an outer surface of an intravenous or arterial catheter. In some embodiments, the medical device surface may include polyurethane or another suitable material.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is partial cutaway view of a portion of an example medical device having layered polymeric coatings, according to some embodiments;

FIG. 1B is an upper perspective view of the medical device of FIG. 1A, according to some embodiments;

FIG. 2 shows daily cumulative release of a water-soluble API for top coatings including different inert polymers; and

FIG. 3 shows daily cumulative release of another water-soluble API for other top coatings including different inert polymers.

### DESCRIPTION OF EMBODIMENTS

In some embodiments, the present disclosure relates to drug-eluting layered polymeric coatings on a medical device surface, aiming to enable sustained therapeutic doses of API release from a coating matrix that traditionally would elicit insufficient API levels. In some embodiments, this is achieved through a multilayer, solvent-cast coating approach, where a primary coating or layer includes a high-concentration, water-soluble or partially water-soluble API within a weak-to-moderate water-swelling polymer. In some embodiments, the primary coating is then over-coated with a top coating that may include an inert, largely nonpolar but water- and API-permeable polymer layer. This configuration may effectively mitigate burst release by controlling water penetration and utilizing pressure to enhance API release rates in a controlled manner, a significant departure from diffusion-only based systems.

In some embodiments, unlike conventional diffusion-driven or single-layer coatings, the devices and methods of the present disclosure employ multiple solvent-cast layers, allowing for enhanced control over mechanical properties, material selection, and API release profiles. Also, the devices and methods of the present disclosure leverage pressure created by the layered polymeric coatings' unique composition and thicknesses to increase the rate of drug release while containing burst release through diffusion limitation. This non-Fickian diffusion mechanism is distinct from Fickian diffusion-based systems and offers a new level of control over drug elution.

In some embodiments, the solvent-cast approach provides greater freedom in choosing polymer, solvent, and API combinations, overcoming limitations related to drug-polymer interactions, charge issues, and temperature sensitivities that are common in other coating methodologies. In some embodiments, the devices and methods of the present disclosure are not solely focused on reducing the total amount of API released but rather on achieving a controlled release mechanism that can be finely tuned through selection of the layered polymeric coatings, namely the primary coating and the top coating. Thus, in some embodiments, the present disclosure provides for application of the layered polymeric coatings to a variety of medical devices, broadening their utility and effectiveness.

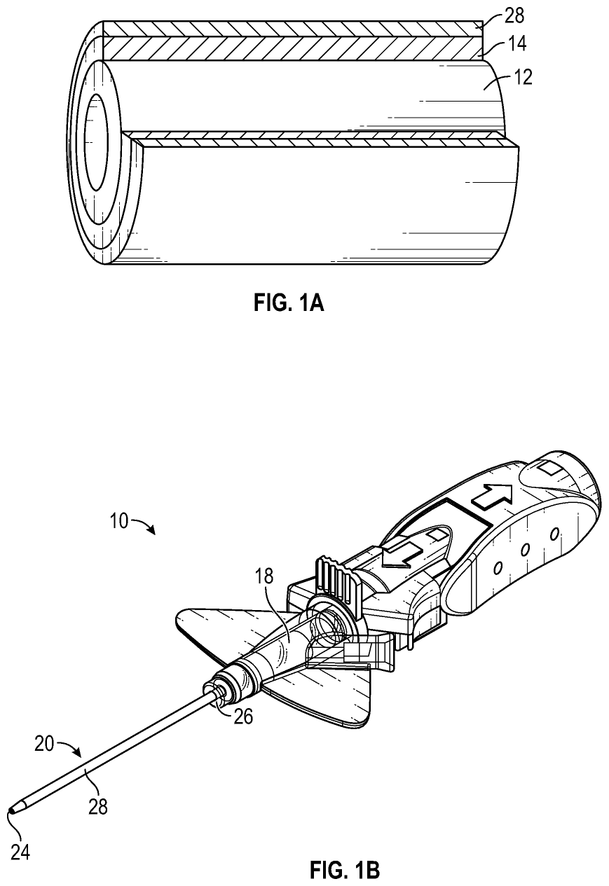

Referring now to FIGS. 1A-1B, in some embodiments, a medical device 10, which may be implantable or indwelling, may include a medical device surface 12. In some embodiments, the medical device 10 may include a primary coating 14 on the medical device surface 12. In some embodiments, the primary coating 14 may include a coating matrix and a water-soluble API within the coating matrix. In some embodiments, the coating matrix may include a hydrophilic polymer, which may swell in response to influx of water. In some embodiments, the water-soluble API may be uniformly dispersed within the coating matrix to form a monolith.

In some embodiments, the medical device surface 12 may include an outer surface of an intravenous or arterial catheter. For example, the medical device 10 may include a catheter system, and the catheter system may include a catheter adapter 18 and a catheter tube 20 extending from a distal end 22 of the catheter adapter 18. FIG. 1A illustrates a middle section of the catheter tube 20 with the primary coating 14 and the top coating 28 partially cutaway for illustrative purposes, according to some embodiments. In some embodiments, the catheter tube 20 may include a distal end 24 and a proximal end 26. In some embodiments, the primary coating 14 and a top coating 28 may extend along all or a portion of a length of the catheter tube 20. In some embodiments, the top coating 28 and the catheter tube 20 may sandwich the primary coating 14 such that an entirety of the primary coating 14 is covered or encapsulated. In some embodiments, the medical device surface 12 may include polyurethane or another suitable material.

In some embodiments, the coating matrix may include a non-ionic polyurethane. In some embodiments, the coating matrix may include an aromatic-polyether polyurethane, an aromatic-polycarbonate polyurethane, an aliphatic-polyether polyurethane, an aliphatic-polycarbonate polyurethane, or another suitable polyurethane. Non-limiting examples of the water-soluble API may include an antimicrobial, antithrombogenic, anti-inflammatory, or an anti-restenosis compound.

In some embodiments, the medical device 10 may include the top coating 28 on the primary coating 14. In some embodiments, the top coating 28 may include an inert polymer, which may facilitate the water-soluble API crossing the top coating when dissolved and released from the primary coating. In used in the present disclosure, the term “inert polymer” refers to a polymer that is non-functionalized, meaning it lacks reactive functional groups that would allow it to undergo chemical reactions under its conditions of use. Because the inert polymer is non-functionalized, this can confer stability and resistance to chemical, thermal, and physical degradation of the inert polymer. The inert polymer can maintain its structural integrity when exposed to an activating condition such as UV light, ozone, or another activating condition for a period up to, for example, several hours.

In some embodiments, the medical device 10 may deliver sustained release of the water-soluble API by non-Fickian diffusion across the top coating 28. In further detail, in some embodiments, in response to water (and/or other dissolution media from a body of a patient) crossing the top coating 28 into the coating matrix, the water-soluble API may be released from the coating matrix and the primary coating may swell to create a pressure that drives the water-soluble API across the top coating 28. In some embodiments, the pressure may drive the water-soluble API across the top coating 28 faster and with more control than diffusion alone. In some embodiments, the pressure created by the swelling of the primary coating 14, in combination with diffusion, causes the API to cross the top coating 28 at a rate greater than expected in Fickian diffusion.

In an osmotic piston-driven drug delivery system, described, for example, in U.S. Pat. No. 6,436,091, entitled “METHODS AND IMPLANTABLE DEVICES AND SYSTEMS FOR LONG TERM DELIVERY OF A PHARMACEUTICAL AGENT,” filed Nov. 16, 1999, a semi-permeable membrane allows entry of water into a housing. Water tends to cross the semi-permeable membrane into an osmotic engine compartment, which includes an osmotic agent. As water crosses the semi-permeable membrane into the osmotic engine compartment, a piston slides, which causes a drug within a reservoir to effuse from a delivery orifice.

In some embodiments, unlike the osmotic piston-driven drug delivery system, the medical device 10 may not include a piston and/or a delivery orifice. Instead, in some embodiments, the top coating 28 may allow both entry of water and exit of the API therethrough, providing a pump across the top coating 28 based on non-Fickian diffusion. In some embodiments, the top coating 28 may form an uninterrupted layer across the medical device surface 12 without any gaps or orifices in the top coating 28.

A semi-permeable membrane, such as in the osmotic piston-driven drug delivery system, allows osmosis or movement of water across the semi-permeable membrane from a first solution into a second solution that is more concentrated than the first solution until the first solution and the second solution have equal concentrations. Solute or pharmacological agents may not move across the semi-permeable membrane. In some embodiments, unlike the osmotic piston-driven drug delivery system, the medical device 10 may not include a semi-permeable membrane. Instead, in some embodiments, the top coating 28 may allow both the water-soluble API and water to cross the top coating 28. In some embodiments, the top coating 28 provides release of the water-soluble API therethrough non-Fickian diffusion based on increased pressure from swelling of the primary coating 14.

In some embodiments, unlike the osmotic piston-driven drug delivery system and other delivery systems known in the art, the medical device 10 may not include a drug reservoir that is an open lumen or cavity and/or contains a solid drug core. Instead, in some embodiments, the water-soluble API may be uniformly or homogenously dispersed in the coating matrix such that the coating matrix is monolithic. In some embodiments, the water-soluble API uniformly dispersed within the coating matrix may facilitate controlled release of the water-soluble API. In some embodiments, the hydrophilic polymer and/or the inert polymer may be non-biodegradable, which may extend a life of the layered polymeric coatings in the body of the patient.

In some embodiments, the water-soluble API may not be covalently or ionically bound to the coating matrix, which may ease release of the water-soluble API from the coating matrix. In some embodiments, the water-soluble API may be physically mixed with a material of the coating matrix, which may include the hydrophilic polymer. In some embodiments, the water-soluble API may be evenly dispersed or encapsulated within the coating matrix and/or release of the water-soluble API may depend on diffusion of the water-soluble API through the coating matrix in response to swelling of the hydrophilic polymer with water.

In these embodiments, the top coating 28 may be hydrophobic or more hydrophobic than the primary coating 14. In some embodiments, the inert polymer of the top coating 28 may include a weak-to-moderate hydrophobic resin such as polyethylene-co-vinyl acetate or another suitable hydrophobic resin. In some embodiments, the weak-to-moderate hydrophobic resin may be only partially soluble in water or may not be readily soluble in water. In some embodiments, the weak-to-moderate hydrophobic resin may be only partially degradable in an aqueous environment or may not be readily soluble in the aqueous environment. In some embodiments, the weak-to-moderate hydrophobic resin may exhibit water swelling properties, meaning the weak-to-moderate hydrophobic resin can absorb water and increase in size. In some instances, the weak-to-moderate hydrophobic resin may experience a mass increase between 0% to 20% in response to exposure to water. In some embodiments, the weak-to-moderate hydrophobic resin may not absorb water, exhibiting a 0% mass change in response to exposure to water.

In some embodiments, the top coating 28 may consist of or be limited only to the inert polymer. In some embodiments, the inert polymer may not include an API. In some embodiments, the inert polymer may not include any ionic content. In some embodiments, the inert polymer may not degrade under physiological conditions. In some embodiments, the inert polymer may exhibit water swelling properties, meaning the inert polymer can absorb water and increase in size. However, the increase in size may be limited to a relatively small range of 1% to 5% of the inert polymer's original volume or mass prior to exposure to water. In some embodiments, the inert polymer may not be reactive to its environment besides the water swelling properties.

In some embodiments, the top coating 28 may not include a gel layer, which may slow penetration of water and place emphasis on whether a particular polymer of the top coating 28 is hydrophobic or hydrophilic as these properties may change a nature of the gel layer.

In some embodiments, the top coating 28 may moderate burst release by limiting diffusion of water from the human body into the primary coating 14. In further detail, in some embodiments, the top coating 28 that is water-restrictive may allow burst release to be reduced or eliminated, preventing acute API toxicity. In some embodiments, the top coating 28 proximate and contacting the primary coating 14 not only serves as a diffusion barrier, preventing rapid swelling of the primary coating 14 and subsequent uncontrolled burst release of the API, the top coating 28 proximate and contacting the primary coating 14 may also induce non-Fickian diffusion across the top coating 28, driving the API across the primary coating 14 at a controlled rate.

In some embodiments, the primary coating 14 and the top coating 28 may be solvent-cast, which may allow for the water-soluble API to be non-covalently incorporated into the coating matrix. In some embodiments, the primary coating 14 and the top coating 28 that are solvent-cast may be less susceptible to API aggregation due to molecular charging and not limited by temperature-related issues, discussed below.

In some embodiments, the primary coating 14 and the top coating 28 may not be melt-cast or electrostatically sprayed. In some instances, electrostatic spraying may lead to an uneven distribution of particles, and the charging process might cause a particular API to accumulate on a particular coating's surface. Also, melt-cast coatings can face temperature limitations. In further detail, APIs that degrade at temperatures lower than a melting point of a particular polymer of the primary coating 14 or the top coating 28 may be unsuitable due to drug degradation. Moreover, solvents that boil at temperatures below the particular polymer's melting point may be unsuitable due to drug degradation.

In some embodiments, a method of preparing layered polymeric coatings on the medical device 10 to deliver sustained release of the water-soluble API by non-Fickian diffusion across the top coating 28 may include obtaining the medical device 10, which may include the medical device surface 12. In some embodiments, the method may include forming the primary coating 14 on the medical device surface 12. In some embodiments, forming the primary coating 14 on the medical device surface 12 may include forming a first solution by dissolving a hydrophilic polymer and the water-soluble API in a first solvent such that the water-soluble API is uniformly dispersed within the coating matrix.

In some embodiments, forming the primary coating 14 on the medical device surface 12 may include solvent-casting the medical device 10 surface in the first solution. In some embodiments, forming the primary coating 14 on the medical device surface 12 may include evaporating the first solvent from the medical device surface 12. In other embodiments, solvent-casting the medical device surface 12 in the first solution may include dip coating the medical device surface 12 in the first solution. In further detail, in some embodiments, the medical device surface 12 may be immersed in the first solution and then withdrawn from the first solution at a controlled rate. In some embodiments, an immersion time of the medical device surface 12 may vary based on a desired thickness of the primary coating and/or properties of the first solution. In some embodiments, the controlled rate at which the medical device surface 12 is withdrawn may vary based on a desired thickness of the primary coating 14 and to maintain a uniform thickness of the primary coating 14.

In some embodiments, the medical device surface 12 may be withdrawn from the first solution at an initial rate of about 117.6 mm/s and a final rate of about 60 mm/s. In these and other embodiments, the primary coating 14 may have a thickness of about 15 micrometers, which may facilitate adequate swelling to facilitate non-Fickian diffusion via increased pressure from swelling of the primary coating 14. In some embodiments, a velocity change of the withdrawal of the medical device surface from the first solution may not be linear with respect to a length of the medical device surface 12. In some embodiments, after solvent-casting the medical device surface 12 in the first solution, the first solvent may be evaporated from the medical device surface 12.

In some embodiments, the method may include forming the top coating 28 on the primary coating 14. In some embodiments, forming the top coating 28 on the primary coating 14 may include dissolving the inert polymer in a second solvent to form a second solution. In some embodiments, forming the top coating 28 on the primary coating 14 may include solvent-casting the medical device surface 12 in the second solution. In some embodiments, solvent-casting may provide flexibility in choosing combinations of the first solvent, the second solvent, the API, the hydrophilic polymer, and the inert polymer, because molecular charging is reduced.

In some embodiments, solvent-casting the medical device surface 12 in the second solution may include dip coating the medical device surface 12 after the primary coating 14 has been applied to the medical device surface 12. In further detail, in some embodiments, the medical device surface 12 may be immersed in the second solution and then withdrawn from the second solution at a controlled rate. In some embodiments, an immersion time of the medical device surface 12 in the second solution may vary based on a desired thickness of the top coating 28 and/or properties of the second solution. In some embodiments, the controlled rate at which the medical device surface 12 is withdrawn from the second solution may vary based on a desired thickness of the top coating 28 and to maintain a uniform thickness of the top coating 28.

In some embodiments, the medical device surface 12 may be withdrawn from the second solution at an initial rate of about 117.6 mm/s and a final rate of about 60 mm/s, or a same initial rate and final rate as withdrawn from the first solution. In these and other embodiments, the top coating 28 may have a thickness of less than 15 micrometers, which may facilitate release of the water-soluble API from the primary coating across the top coating 28. In some embodiments, a velocity change of the withdrawal of the medical device surface 12 from the second solution may not be linear with respect to a length of the medical device surface 12. In some embodiments, after solvent-casting the medical device surface 12 in the second solution, the second solvent may be evaporated from the medical device surface 12.

In some embodiments, because the primary coating 14 is applied by dip coating, an increased thickness of the primary coating 14 may correspond to a higher quantity of the water-soluble API. In some embodiments, the thickness of the primary coating 14 may be selected based on a desired initial loading of the water-soluble API. In some embodiments, the thickness of the primary coating 14 and the thickness of the top coating 28 may vary based on a desired gauge of a catheter that includes the top coating 28 and the primary coating 14. For example, the thickness of the primary coating 14 and the thickness of the top coating 28 may be selected to achieve a particular total outer diameter, which may not exceed a size that would no longer be classified as the desired gauge. In some embodiments, the thickness of the primary coating 14 and the thickness of the top coating 28 may affect how far the water-soluble API has to travel to be released and thus may have a minor effect on a cumulative release profile of the water-soluble API.

In some embodiments, the first solvent may include a polar organic solvent. In some embodiments, the hydrophilic polymer may be non-ionic. In some embodiments, the second solvent may include a non-polar organic solvent. In some embodiments, the non-polar organic solvent may include toluene or another suitable non-polar organic solvent. In some embodiments, the inert polymer of the top coating 28 may include polyethylene-co-vinyl acetate. In some embodiments, the polyethylene-co-vinyl acetate ranging from 12% to 40% vinyl acetate may be dissolved in a non-polar organic solvent such as toluene to form the second solution.

In some embodiments, the coating matrix may include a weak-to-moderate water-swelling polymer. In some embodiments, the coating matrix may include polyurethane, which may be non-ionic. In some embodiments, the coating matrix may include an aromatic-polyether polyurethane, an aromatic-polycarbonate polyurethane, an aliphatic-polyether polyurethane, or an aliphatic-polycarbonate polyurethane, or another suitable polyurethane. In some embodiments, a hydrophilicity of the coating matrix and/or the top coating 28 measured by mass change due to water uptake may vary. In some embodiments, the polyurethane of the coating matrix may range from 0.75% mass change due to water uptake up to 98.6% mass change due to water uptake.

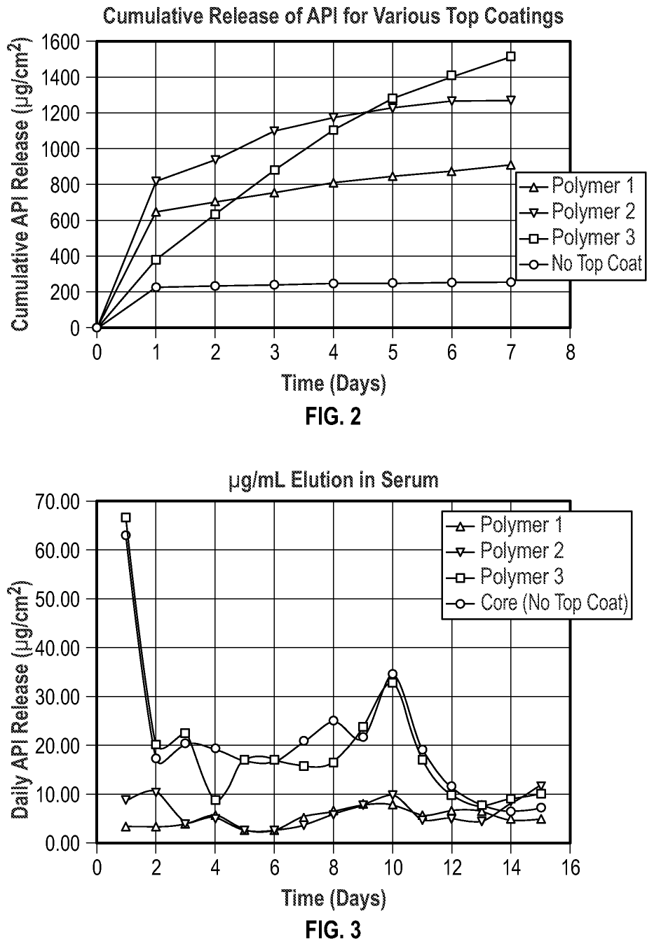

Referring now to FIG. 2, daily cumulative release of the water-soluble API is illustrated for top coatings including different inert polymers. “Polymer 1” in FIG. 2 corresponds to a top coating including a first inert polymer. “Polymer 2” in FIG. 2 corresponds to a top coating including a second inert polymer. “Polymer 3” in FIG. 2 corresponds to a top coating including a third inert polymer. “No Top Coat” in FIG. 2 indicates only a primary coating without a top coating was applied. Each of Polymer 1, Polymer 2, and Polymer 3 were applied to the primary coating on a medical device surface, which was a same primary coating for each of Polymer 1, Polymer 2, Polymer 3, and No Top Coat. The top coating of Polymer 1, Polymer 2, or Polymer 3 may include or correspond to the top coating 28 of FIG. 1, and the primary coating may include or correspond to the primary coating 14 of FIG. 1.

In the example shown in FIG. 2, the first inert polymer of Polymer 1 was BIONATE® medical grade thermoplastic polycarbonate polyurethane (PCU) 80A manufactured by DSM Biomedical Inc., the second inert polymer of Polymer 2 was TECOFLEX™ medical grade aliphatic polyether-based thermoplastic polyurethane (TPU) 80A manufactured by Lubrizol Corporation, and the third inert polymer of Polymer 3 was PT83-100 polymer with BIONATE® PCU 80A. PT-83-100 polymer corresponds to PATHWAYS™ TPU manufactured by Lubrizol Corporation having a Shore A hardness of 83 (similar to Polymers 1 and 2) and allowing up to 100% swelling in water.

As illustrated in FIG. 2, addition of a particular top coating that includes an inert polymer increases a total amount of the water-soluble API released over a 7-day period. By varying a composition of the particular top coating, control of a release rate of the water-soluble API is achieved. Near zero-order release of the water-soluble API (i.e., near constant release over time, shown by a nearly linear cumulative release profile) is achieved with Polymer 3. In some embodiments, the polyurethane of the coating matrix may range from about 5% mass change due to water uptake up to about 50% mass change due to water uptake, which may facilitate a more linear cumulative release profile. In some embodiments, the polyurethane of the coating matrix may range from about 10% mass change due to water uptake up to about 20% mass change due to water uptake, which may also facilitate a more linear cumulative release profile.

A coating matrix used in the experiment of FIG. 2 included a weakly hydrophilic aromatic-polycarbonate polyurethane. The “No Top Coat” the primary coating quickly reaches its API-polymer equilibrium and release of the water-soluble API halts after a short period of time. However, applying a top coating to the primary coating significantly changes cumulative release of the water-soluble API by provoking non-Fickian diffusion and an osmotic pump effect that constantly drives the water-soluble API from the primary coating and across the top coating.

Referring now to FIG. 3, daily cumulative release of the water-soluble API is illustrated for top coatings including different inert polymers than Polymer 1, Polymer 2, and Polymer 3 of FIG. 2. The water-soluble API used in FIG. 3 is also different from FIG. 2. A separate chemistry set was selected for the experiment of FIG. 3 compared to FIG. 2 to demonstrate how different water-soluble APIs can behave in different applications.

“Polymer 1” in FIG. 3 corresponds to a top coating including a first inert polymer. “Polymer 2” in FIG. 3 corresponds to a top coating including a second inert polymer. “Polymer 3” in FIG. 3 corresponds to a top coating including an identical composition as a primary coating. As such, there the osmotic pump effect is not generated for Polymer 3. “Core (no top coat)” in FIG. 3 indicates only the primary coating without a top coating was applied. Each of Polymer 1, Polymer 2, and Polymer 3 were applied to the primary coating on a medical device surface, which was a same primary coating for each of Polymer 1, Polymer 2, Polymer 3, and Core (no top coat). The top coating of Polymer 1, Polymer 2, or Polymer 3 may include or correspond to the top coating 28 of FIG. 1, and the primary coating may include or correspond to the primary coating 14 of FIG. 1. In the example shown in FIG. 3, the primary coating was TECOFLEX™ aliphatic polyether-based thermoplastic polyurethane (TPU) 80A, which was also the top coating of Polymer 3. The first inert polymer of Polymer 1 was polyethylene-co-vinyl acetate (12% vinyl acetate), and the second inert polymer of Polymer 2 was polyethylene-co-vinyl acetate (40% vinyl acetate).

As illustrated in FIG. 3, addition of different inert polymers as the top coating drastically reduces both a day 1 burst release as well as a day-to-day release. Also, the osmotic pump effect across the top coatings of Polymer 1 and Polymer 2 long delays an equilibrium state of the medical device surface. After day 10, daily release of the water-soluble API continues to decline as the equilibrium state is reached for Polymer 3 and Core (no top coat). However, Polymer 1 and Polymer 2 continue to see near zero-order release at a prolonged period of time with signs of increasing daily release of the water-soluble API instead of a typical decline.

By varying a composition of the top coating, control over a release rate of the water-soluble API and burst release is achieved. The coating matrix in FIG. 3 included aliphatic-polyether polyurethane, which is more hydrophilic than the aromatic-polycarbonate polyurethane used in the experiment of FIG. 2. The first inert polymer and the second inert polymer are different formulations of polyethylene-co-vinyl acetate resin. The first inert polymer and the second inert polymer may be more hydrophobic than the primary coating, which allows the burst release to be drastically reduced while still benefiting from the primary coating swelling overtime to facilitate non-Fickian diffusion.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. An implantable or indwelling medical device to deliver sustained release of an active pharmaceutical ingredient (API) by non-Fickian diffusion across a top coating, the implantable or indwelling medical device comprising:
a medical device surface;
a primary coating on the medical device surface, wherein the primary coating comprises:
a coating matrix; and
a water-soluble API within the coating matrix; and
wherein the top coating is disposed on the primary coating, wherein the top coating comprises an inert polymer,
wherein in response to water crossing the top coating into the coating matrix, the water-soluble API is released from the coating matrix and the primary coating swells to create a pressure that drives the API across the top coating.

2. The implantable or indwelling medical device of claim 1, wherein the primary coating and the top coating are solvent-cast.

3. The implantable or indwelling medical device of claim 1, wherein the water-soluble API is uniformly dispersed in the coating matrix, wherein the water-soluble API is not covalently or ionically bound to the coating matrix.

4. The implantable or indwelling medical device of claim 1, wherein the inert polymer of the top coating comprises polyethylene-co-vinyl acetate.

5. The implantable or indwelling medical device of claim 1, wherein the coating matrix comprises a non-ionic polyurethane.

6. The implantable or indwelling medical device of claim 5, wherein the coating matrix comprises an aromatic-polyether polyurethane, an aromatic-polycarbonate polyurethane, an aliphatic-polyether polyurethane, or an aliphatic-polycarbonate polyurethane.

7. The implantable or indwelling medical device of claim 1, wherein the primary coating has a thickness of at least 15 micrometers.

8. The implantable or indwelling medical device of claim 1, wherein the top coating has a thickness less than 15 micrometers.

9. The implantable or indwelling medical device of claim 1, wherein the medical device surface comprises an outer surface of an intravenous or arterial catheter.

10. The implantable or indwelling medical device of claim 7, wherein the medical device surface comprises polyurethane.

11. A method of preparing layered polymeric coatings on an implantable or indwelling medical device to deliver sustained release of an active pharmaceutical ingredient (API) by non-Fickian diffusion across a top coating, the method comprising:
obtaining an implantable or indwelling medical device comprising a medical device surface;
forming a primary coating on the medical device surface, comprising:
forming a first solution by dissolving a hydrophilic polymer and a water-soluble API in a first solvent;
solvent-casting the medical device surface in the first solution; and
evaporating the first solvent from the medical device surface to form a coating matrix and the water-soluble API within the coating matrix, wherein the water-soluble API is uniformly dispersed in the coating matrix; and
forming a top coating on the primary coating, comprising:
dissolving an inert polymer in a second solvent to form a second solution;
solvent-casting the medical device surface having the primary coating on the medical device surface in the second solution; and
evaporating the second solvent from the medical device surface,
wherein in response to water crossing the top coating into the coating matrix, the water-soluble API is released from the coating matrix and the primary coating swells to create a pressure to drive the API across the top coating.

12. The method of claim 11, wherein the first solvent comprises a polar organic solvent.

13. The method of claim 11, wherein the hydrophilic polymer is non-ionic.

14. The method of claim 11, wherein the second solvent comprises a non-polar organic solvent.

15. The method of claim 14, wherein the non-polar organic solvent comprises toluene.

16. The method of claim 11, wherein the inert polymer of the top coating comprises polyethylene-co-vinyl acetate.

17. The method of claim 11, wherein the coating matrix comprises a non-ionic polyurethane.

18. The method of claim 17, wherein the coating matrix comprises an aromatic-polyether polyurethane, an aromatic-polycarbonate polyurethane, an aliphatic-polyether polyurethane, or an aliphatic-polycarbonate polyurethane.

19. The method of claim 11, wherein the medical device surface comprises an outer surface of an intravenous or arterial catheter.

20. The method of claim 19, wherein the medical device surface comprises polyurethane.
